(19) United States
(12) Patent Application Publication
(10) Pub. No.: US 2015/0177227 A1
(43) Pub. Date: Jun. 25, 2015

(54) CELLULAR AND ANIMAL MODELS FOR SCREENING THERAPEUTIC AGENTS FOR THE TREATMENT OF ALZHEIMER'S DISEASE

(71) Applicant: Jordan L. HOLTZMAN, Minneapolis, MN (US)

(72) Inventor: Jordan L. Holtzman, Minneapolis, MN (US)

(21) Appl. No.: 14/414,650

(22) PCT Filed: Jul. 15, 2013
(86) PCT No.: PCT/US13/50532

§ 371 Date: Jan. 13, 2015

Related U.S. Application Data
(60) Provisional application No. 61/671,437, filed on Jul. 13, 2012.

(51) Int. Cl. G01N 33/50; C12N 9/10

(52) U.S. Cl. CPC G01N 33/5073; C12N 9/1048; G01N 2800/2821; C07K 2319/60; G01N 2500/10

(57) ABSTRACT

Constructs, models, and methods to assay candidate therapeutic agents to treat Alzheimer's disease are described herein.

### BACKGROUND

Alzheimer's disease is a major cause of morbidity and mortality in the elderly. It is characterized by dementia associated with deposits of a 42 amino acid peptide, β-amyloid, in the extracelluar space of the cerebral cortex. The dominant model for the etiology of the dementia is that it is secondary to the neurotoxicity of this peptide, either in the form of deposits termed plaques or soluble aggregates. Based on this paradigm a number of investigators have developed transgenic mouse models that exhibit both plaque deposits and decreased cognitive function with age. These models have been employed for screening of potential therapeutic agents for efficacy.

At least seven agents that were effective in these mouse models proved to be ineffective when tested in well designed, phase III clinical trials (Table 1). All of these agents were identified as a result of their ability to clear plaque and reduce the cognitive decline seen in these mouse models.

TABLE 1
List of Failed Phase 3 Trials of Agents which
were Effective in Transgenic Mouse Models

| Drug | Mechanism | Reference | Animal References |
|---|---|---|---|
| Abeta Vaccine | Clear abeta | Holmes | Janus et al. 2000; |
| | | et al. 2008 | Morgan et al. 2000 |
| Tarenflurbil | γ-secretase | Green et al. | Kukar et al. 2007 |
| | inhibitor | 2009 | |
| Semagacestat | γ-secretase | Schor 2011 | Abramowski et al. 2008 |
| | inhibitor | | |
| MK-677 | IGF-1 | Sevigny | Carro et al. 2002 |
| | Secretagogue | et al. 2008 | |
| Ginkgo biloba | Antioxidant | DeKosky | Stackman et al. 2003 |
| | | et al. 2008 | |
| Estrogen | Hormone | Espeland | Levin-Allerhand et al. |
| | Replacement | et al. 2004 | 2002; Carroll et al. 2007 |
| Docosahexaeonic | Omega-3 | Quinn | Calon et al. 2004 |
| Acid | Fatty Acid | et al. 2010 | |

In the next year the results of ongoing phase III clinical trials of the humanized antibodies bapineuzumab from Johnson & Johnson/Pfizer and solanezumab from Eli Lilly & Co., both of which have been shown to clear plaque, should help to determine whether β-amyloid has any role in the cognitive decline. If, like the vaccine trial, the humanized antibodies fail to show any benefit, it would be difficult to continue to accept that β-amyloid has a major role in the etiology of Alzheimer's disease associated dementia. Even if these studies demonstrate some statistically significant benefit, it is unlikely to have a major clinical impact since neither antibody showed any trend in phase II clinical trials.

There are problems with the β-amyloid model. These include a poor correlation between plaque burden and the development of dementia; whether β-amyloid is neurotoxic; whether the deposits or soluble aggregates are the toxic agent; the issue of whether the transgenic mice are a valid model for the disease; and whether these trials failed because the patients' disease had progressed to a point where they could no longer benefit from therapy (Gandy, 2005; Seabrook et al. 2005; Robakis, 2010).

In view of the foregoing, there is a need for an appropriate cellular and/or animal model to screen drugs for treating Alzheimer's disease.

### SUMMARY

Constructs, models, and methods to assay candidate therapeutic agents to treat Alzheimer's disease are described herein. In an embodiment, a construct includes a polynucleotide encoding a fusion protein of (a) a chaperone protein or monosaccharide transferase (MST) and (b) a fluorescent protein. In an embodiment, the fusion occurs where at least one exon of a polynucleotide encoding a chaperone protein or MST is followed by a polynucleotide encoding a fluorescent protein. In an embodiment, the 3′ end of a polynucleotide encoding a fluorescent protein begins at the 5′end of the last exon of a chaperone protein or MST. In an embodiment, a construct comprises a polynucleotide encoding ERp57 and a polynucleotide encoding a fluorescent protein. In an embodiment, a construct comprises a polynucleotide encoding ERp57 and a polynucleotide encoding green fluorescent protein.

In an embodiment, a cellular model includes a plurality of cells comprising a construct as disclosed herein. In an embodiment, an animal model includes a cell comprising a construct as disclosed herein. Methods are disclosed of administering candidate therapeutic agent and measuring fluorescence in cellular models.

### DESCRIPTION

### Definitions

The term “normal chaperone levels” refers to the mean concentration of chaperones that can typically be found in cerebrospinal fluid from a control population. Suitable control populations include, for example, young people, elderly people without Alzheimer's disease, and the like. A normal chaperone level can be about 27±0.2 ng/ml.

The terms “chaperone” or “chaperone protein” or “chaperonin” refers to proteins that catalyze folding, formation of tertiary structure, formation of quaternary structure, and/or other processing to make an active protein. As described herein, several chaperones can decline in level with age and can be correlated with age-related diseases and disorders. In particular, tissue levels of one or more of the chaperones BiP, calreticulin, calnexin, Erp72, Q2, and Q5 can decrease with age of a mammal, such as a rodent or a human, and can correlate with a disease. Chaperones include a family known as a thiol:protein disulfide oxidoreductase (TPDO). A TPDO represents a preferred chaperone of the present invention. A preferred TPDO is TPDO-Q2. TPDO-Q2 has also been called ERp57 and GRp58. As used herein, chaperone refers to any of the common names for these proteins and all naturally occurring variant forms of this protein, including glycosylated and nonglycosylated forms.

The term “exon” refers to a region of a gene that has intervening sequences (introns) where the exonic DNA is actually translated or expressed. In contrast, introns are not translated or expressed but rather spliced out of the mRNA.

The term “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins (e.g., an antibody or antibody fragment), peptides, hormones, nucleic acids, gene constructs and the like.

The terms “candidate therapeutic agent” or “candidate therapeutic drug” refer to a therapeutic agent as defined above when tested and/or characterized in a cellular or animal model as disclosed herein to determine its effect on expression of a chaperone protein or MST.

The term “mammal” for purposes of treatment or therapy refers to any animal classified as a mammal, including humans, domestic and farm animals, and zoo, sports, or pet animals, such as dogs, horses, cats, cows, and the like.

A “control” is an alternative subject or sample used in an analytical procedure for comparison purposes. A control can be “positive” or “negative”. For example, where the purpose of an analytical procedure is to detect a differentially expressed transcript or polypeptide in cells or tissue affected by a disease of concern, it is generally preferable to include a positive control, such as a subject or a sample from a subject exhibiting the desired expression and/or clinical syndrome characteristic of the desired expression, and a negative control, such as a subject or a sample from a subject lacking the desired expression and/or clinical syndrome of that desired expression.

The term “treatment” or “treating” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. For the purposes herein, “treatment” does not include “preventing” or “prevention” or “prophylaxis”.

The term “label” when used herein refers to a detectable compound or composition that is fused to a protein. The label may be detectable by itself (e.g. radioisotope labels or fluorescent labels) or, in the case of an enzymatic label, may catalyze chemical alteration of a substrate compound or composition that is detectable (e.g., avidin-biotin).

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label.

“Polypeptide” refers to a peptide or protein containing two or more amino acids linked by peptide bonds, and includes peptides, oligimers, proteins, and the like. Polypeptides can contain natural, modified, or synthetic amino acids. Polypeptides can also be modified naturally, such as by post-translational processing, or chemically, such as amidation, acylation, cross-linking, and the like.

The terms “antisense molecule” or “antisense RNA” or “antisense reagent” refer to a polynucleotide that is a complement to a message (or “sense”) strand of RNA. The antisense molecule can form a duplex with a sense strand of RNA (mRNA). This duplex can block translation of the mRNA into a polypeptide by blocking a ribosome's access to the mRNA or a RNA duplex can be degraded by ribonucleases.

The terms “biological sample” refers to all biological fluids and excretions isolated from any given subject. In the context of the invention such samples include, but are not limited to, blood and fractions thereof, blood serum, blood plasma, urine, excreta, semen, seminal fluid, seminal plasma, prostatic fluid, pre-ejaculatory fluid (Cowper's fluid), pleural effusion, tears, saliva, sputum, sweat, biopsy, ascites, cerebrospinal fluid, amniotic fluid, lymph, marrow, cervical secretions, vaginal secretions, endometrial secretions, gastrointestinal secretions, bronchial secretions, breast secretions, ovarian cyst secretions, or hair, as well as tissue extracts such as homogenized tissue, and cellular extracts.

The term “detecting” is used in the broadest sense to include both qualitative and quantitative measurements of a specific molecule, for example, measurements of a specific molecule such as a chaperone protein.

### Chaperone Protein

Chaperones catalyze folding, formation of tertiary structure, formation of quaternary structure, and/or other processing to make an active protein. As described previously, several chaperones can decline in level with age and can be correlated with age-related diseases and disorders. Chaperones include a family known as a thiol:protein disulfide oxidoreductase (TPDO). A TPDO represents a preferred chaperone. A preferred TPDO is TPDO-Q2. TPDO-Q2 has also been called ERp57 and GRp58. In an embodiment, a chaperone protein is an ER chaperone protein. In an embodiment, an ER chaperone protein is ERp57, GRp78, GRp94, GRp104, PDI, calnexin, calreticulin, or ERp72 and all naturally occurring variant forms, including glycosylated and nonglycosylated forms.

Chaperones are, in general, well studied and/or characterized proteins. Well characterized features of numerous chaperones include the genes encoding them in organisms ranging from bacteria to humans, recombinant expression systems (e.g., vectors, plasmids, and the like) for these proteins, methods of producing these proteins, protein sequences and structures, certain protein substrates, and certain biological functions.

### Animal and Cellular Models

Due to the lack of success of drugs to treat Alzheimer's disease in phase III clinical trials after success in transgenic mouse models, a new cellular and animal model is needed to screen drugs to treat Alzheimer's disease. Disclosed herein are animal and cellular models based on chaperone complexing with β-amyloid for normal posttranslational processing of proteins in the endoplasmic reticulum (ER). In embodiments disclosed herein, methods detect cellular and whole body chaperone levels. In an aging model, chaperone levels decline. Candidate therapeutic agents are administered to cellular and animal models disclosed herein. Successful therapeutic agents raise chaperone levels to increase chaperone/β-amyloid complexes, whereby the complexes allow for normal posttranslational processing of β-amyloid in the ER.

### Cellular Model

In an embodiment, a construct includes a polynucleotide encoding a fusion protein of (a) a chaperone protein or monosaccharide transferase (MST) and (b) a detectable label such as a fluorescent protein. In an embodiment, the fusion occurs where at least one exon of a polynucleotide encoding a chaperone protein or MST is followed by a polynucleotide encoding a fluorescent protein. In an embodiment, the 3′ end of a polynucleotide encoding a fluorescent protein begins at the 5′end of the last exon of a chaperone protein or MST. Thereby, the fusion occurs between the 5′ end encoded by an exon of a chaperone or MST and a fluorescent protein. In an embodiment, a construct comprises a polynucleotide encoding ERp57 and a polynucleotide encoding a fluorescent protein. In an embodiment, a construct comprises a polynucleotide encoding at least one chaperone protein or monosaccharide transferase (MST) and a polynucleotide encoding green fluorescent protein (GFP). In an embodiment, a construct comprises a polynucleotide encoding ERp57 and a polynucleotide encoding green fluorescent protein. In an embodiment, a construct comprises a polynucleotide encoding monosaccharide transferase (MST) and a polynucleotide encoding green fluorescent protein. In an embodiment, a construct comprises a polynucleotide encoding at least one chaperone protein or monosaccharide transferase (MST) and a polynucleotide encoding green fluorescent protein, wherein the GFP is fused to ERp57 following a portion encoded by an exon.

In an embodiment, a cell comprises a construct as described herein.

In an embodiment, a cellular model includes a plurality of cells comprising a construct comprising a polynucleotide encoding at least one chaperone protein or monosaccharide transferase (MST) and a polynucleotide encoding a fluorescent protein. In an embodiment, the fusion protein comprises a fluorescent protein fused to a chaperone protein or MST, wherein the fluorescent protein is fused to a chaperone protein (e.g., ERp57) or a MST. Thereby, a cellular model includes a polynucleotide construct that encodes a fusion protein comprising a chaperone and a fluorescent protein. A cellular model also includes a polynucleotide construct that encodes a fusion protein comprising a MST and a fluorescent protein. In an embodiment, a cellular model comprises a polynucleotide construct encoding an ERp57::GFP fusion protein, wherein the polynucleotide encoding the GFP is fused to an exon of ERp57. In an embodiment, a cellular model includes a cell comprising any of the constructs disclosed herein.

A cellular model includes a plurality of cells comprising a construct as disclosed herein. A cellular model can be an in vitro collection of cells used for assays. In an example, a cellular model includes a plurality of cells comprising a construct as disclosed herein in a multiwell cell culture plate. A candidate therapeutic agent can be applied to each well of multiwell cell culture plate (or microtiter plate) and fluorescence can be detected following an incubation time period or time periods. A cellular model allows high throughput drug screening and includes mammalian cells comprising exons of chaperones (e.g., ERp57) fused with a fluorescent protein. In an embodiment, cells are incubated with a candidate therapeutic agent and screened for increased fluorescence compared to a negative control. In an embodiment, a negative control is vehicle alone (i.e., the solution applied to the cells without the candidate therapeutic agent). In an embodiment, cells are incubated in a microtiter plate and then fluorescence is measured by a microtiter plate reader (e.g., BioTek® microplate readers, Winooski, Vt.). Other chemical and physical techniques to measure fluorescence are well known.

Commonly used fluorescent proteins such as, but not limited to, GFP, RFP, CFP, YFP and mCherry, are compact proteins that can be inserted into an appropriate exon or fused to an appropriate exon do not interfere with normal functions of proteins in cellular metabolism. Fluorescent proteins include, but are not limited to, green fluorescent protein (GFP), red fluorescent protein (RFP), cyan fluorescent protein (CFP), yellow fluorescent protein (YFP), and mCherry fluorescent proteins. Typically, a polynucleotide encoding a fluorescent protein is inserted upstream of the first exon or downstream of the last exon. This will produce a fusion protein (e.g., ERp57::GFP) that does not interfere with function and allows cellular viability.

In an embodiment, a cellular model includes mammalian cells comprising at least one chaperone protein or monosaccharide transferase (MST) fused to a fluorescent protein. In an embodiment, a cellular model includes a polynucleotide encoding a fluorescent protein inserted adjacent to a chaperone or MST gene. In an embodiment, the gene includes introns and/or various control regions including the promoter region that precedes the first exon and the region that follows the last exon, the 3′untranslated region (3′-UTR). In an embodiment, a cellular model includes mammalian cells comprising two or more of a) chaperone proteins, b) MSTs or c) both a chaperone protein and a MST fused to a fluorescent protein.

Mammalian cells include, but are not limited to, cardiac progenitor cells, skeletal muscle cells, islet cells, kidney cells, stem cells, neural cells, or any other cells. Stem cells can be embryonic stem cells. Stem cells can be neural stem cells. Mammalian cells can be neuronal cells, and more particularly embryonic stem cell derived neuronal cells. Mammalian cells can be microglia. Suitable mammalian cells include, but are not limited to Chinese hamster ovary (CHO) cells, baby hamster kidney (BHK) cells, human HeLa cells, PC12 cells, human neuronal (HN) cells, monkey COS-1 cell, and human embryonic kidney 293 cells.

When the construct is activated by a candidate therapeutic agent, a cell synthesizes the chaperone protein or MST fused to a detectable label. For example, stimulated synthesis of luciferase can be detected by bioluminescence after the addition of luciferin and ATP. Similarly, increased production of galactosidases can be determined by the addition of X-gal to medium, which is then hydrolyzed to give a blue color that can then be quantitated by standard spectrophotometric techniques. Finally, an increase in fluorescent proteins can be determined by standard fluorometry. All of these assays can be performed in microtiter plate readers and thereby facilitate high throughput screening for drug discovery.

In rapid drug high throughput screening (HTS) studies, transfected cells can be grown on microtiter plates. Cells should be adherent to a plate. Microtiter plate may be bare plastic or coated with collagen, fibronectin, Matrigel™ (BD Biosciences, San Jose, Calif.), a 3-dimensional substrate, or any other coating appropriate for a particular cell line. It is often beneficial to include nonfluorescent support mesenchymal cells as feeder cells. Various levels of candidate therapeutic agents in an appropriate solvent, such as DSMO, are added, and fluorescence is measured at zero time to determine whether there is quenching of the fluorescence. Optical density can also be determined to ascertain whether a candidate therapeutic agent is absorbing either the excitation or emission wavelengths. These two tests can be used to correct fluorescent measurements after the cells are incubated. Cells as disclosed herein are then incubated for an appropriate time interval and fluorescence is determined. Fluorescence of cells with an administered candidate therapeutic agent can be compared to cells treated with only the vehicle. Differences can be used as a measure of the ability of a candidate therapeutic agent to induce production of target proteins. Results of fluorescent screening assays can be validated by immunological procedures as noted above. Those candidate therapeutic agents showing stimulation of the synthesis of target protein(s) can then be tested in animal models. An earlier study suggests that methoxychlor can be used as a positive control (Morrell et al., 2000)

### Animal Models

Fluorescent models. Embodiments also include non-human animal models based on the same paradigm as the cellular models disclosed herein. In an embodiment, a non-human animal (e.g., a mouse or rat) is transfected with a plasmid containing a conditional promoter and the sequence for small RNAs directed to the mRNAs of a chaperone and/or a MST. Since both chaperone and MST knockouts are lethal mutants, a transcript would have to be activated in mature animals by the administration of an agent (e.g., doxycycline) for cognitive testing. In another embodiment, an antisense reagent targeting a chaperone or MST is administered. Unlike small RNAs, an antisense reagent does not require further processing, thereby circumventing the possibility that the candidate therapeutic agent is blocking maturation of small RNAs rather than enhancing transcription of the target proteins.

In an embodiment, an animal model includes a mammalian laboratory animal (e.g., a mouse or rat) comprising at least one gene for a chaperone protein or a MST fused to a polynucleotide coding for a fluorescent protein. In an embodiment, the gene includes introns and/or various control regions including the promoter region that precedes the first exon and the region that follows the last exon, the 3′untranslated region (3′-UTR). In an embodiment, an animal model includes a mammalian laboratory animal (e.g., a mouse or rat) comprising at least one exon for a chaperone protein or a MST fused to a polynucleotide coding for a fluorescent protein.

In an embodiment, a method of screening one or more candidate therapeutic agent(s) includes administering a candidate therapeutic agent to a non-human animal model described herein, and quantifying an amount of chaperone, MST, OST, or both in a biological sample from a non-human animal model. A chaperone can be a homolog of human ERp57. A biological sample can be cerebrospinal fluid (CSF). A biological sample can be a tissue extract. A tissue extract can be from a live animal or can be upon necropsy. Fluorescence can be measured by well known methods.

Knockouts. It is not possible to develop complete knockout models for ER chaperone proteins or MSTs because such knockouts are embryonic lethals. Furthermore, it would not be possible to determine whether a specific agent enhances transcription and translation of a target protein since a full knockout would lack the target gene. In an embodiment, an animal model includes a laboratory strain of a non-human animal (e.g., mouse or rat) comprising a heterozygotic knockout of a chaperone or MST. In an embodiment, an animal model includes a laboratory strain of a non-human animal (e.g., mouse or rat) comprising a heterozygotic knockout of a homolog of human ERp57. These animals may have only about half the content of the chaperones, MSTs, and oligosaccharide transferases (OST) found in young animals. In the case of chaperone content, this is equivalent to the decline observed in levels of ER chaperones between young and old animals (Erickson et al. 2006).

Instead of embryonically knocking out a target gene, it is preferable to knockout target genes in an adult animal using a cre-lox system and a conditional promoter, which is activated by such agents as tetracycline, tamoxifen, insect juvenile hormone, et al. See, Orban, et al., Proc. Natl. Acad. Sci. USA (1992) 8: 6861-6865, for a review of Cre/lox technology, and Brand and Dymecki, Dev. Cell (2004) 6(1):7-28. In this embodiment, a target gene is transfected with lox sequences at the 5′ and 3′ regions encompassing a set of exons. The animals are also transfected with the cre gene attached to a conditional promoter. On treatment with the agent (e.g., tetracycline, et al.) that activates the promoter to produce cre, the DNA sequence of a target gene between the lox sequences is excised. If the lox genes are appropriately placed, the gene cannot be transcribed to produce an active mRNA. A major advantage of this approach is that it possible to construct the cre promoter so that it is only activated in a single cell type, such as neurons, cardiac myocytes, renal cells, pancreatic β-cells, all the various subsets of lymphocytes and endothelial cells as well as others cells from other tissue types. These animal constructs would also serve as proof of principal model systems. For example, in validating the hypothesis that the loss of cognitive function is due to a decline in the posttranslational processing of proteins in the ER, the animals' cognitive and motor skills can be tested by standard psychomotor procedures both before and after the cre gene is activated to form the heterozygote mutant. Similarly, it could also be used to determine whether test proteins have a role in the loss of immunological function, cardiac function, kidney and insulin production and the decrease in other tissue functions seen with aging.

Small RNA antisense treatment. Cells contain a set of genes that code for small, regulatory RNA molecules. Transfection of synthetic constructs of small RNAs into cells and animals have been extensively used to knock down synthesis of target proteins. In adult cells, the most commonly employed constructs in knock down studies are microRNAs (miRNA) and short hairpin RNAs (siRNA). These RNAs control both transcription and translation of target proteins. In controlling translation their primary target is the mRNA which they bind to and silence. Synthesis of small RNAs into their active forms is a complex process which involves multiple steps and alternative pathways (Czech and Hannon 2011). Small RNAs can regulate unpacking of the DNA-histone complexes as part of the process of activating a target gene (Djupedal and Ekewall, 2009).

Sequences for small RNAs can be transfected as a plasmid containing a DNA construct for the RNA and a reporter gene such as galactosidase. A reporter can be used to confirm that a plasmid has been transfected. A plasmid can also include a conditional promoter activated by agents such as tetracycline, tamoxifen, insect juvenile hormone or any other agents for which there is a conditional promoter available.

Another common procedure for knocking down a specific gene is to expose cells or intact animals to an Adenovirus associated virus (AAV) containing appropriate sequence for small RNAs (Camero et al. 2011). Lentiviruses have also been commonly employed as a vector. A disadvantage with the viral transfection systems is that the liver frequently takes up the bulk of these viral constructs. A further disadvantage with these approaches is that any candidate therapeutic agent may appear to be effective in the cell screening studies but may act primarily by inhibiting one of the steps necessary for processing of a construct to form an active blocking small RNAs. This can lead to a possible false positive result.

Synthetic antisense reagents. Another approach to knocking down synthesis of specific proteins is administering synthetic antisense reagents. The two most commonly used, commercially available agents are the 2′-O-methoxyethyl phosphorothioate and the morpholino antisense constructs. Since these reagents do not readily penetrate the cell membrane, they are routinely used in combination with a permeabilizing agent in cell culture. Yet in both mouse and human in vivo studies, it has been found that high doses of these reagents alone can penetrate into a cell and show activity. Most of the reagents in these studies have been for the treatment of malignancy, but more recently they have been found to be useful in the treatment of Duchenne muscular dystrophy both in mice (Wu et al., 2011) and in humans (Cirak et al., 2011). Administration of these agents would appear to be the simplest approach to knocking down specific chaperones, MSTs, and OSTs. A disadvantage is a lack of tissue specificity. Hence, target proteins would be knocked down in a wide range of tissues. This could cause problems with psychomotor testing since any poor response could be due to decreased skeletal muscle function rather than loss of cognitive function. For example, the water maze is a commonly used procedure to determine the ability of an animal to find a platform as measure of changes in memory. If the reagent causes a reduction in the animal's swimming capacity due to loss of skeletal muscle mass, the animal may not be able to swim long enough to find the platform.

1. A construct comprising a polynucleotide encoding a fusion protein of (a) a chaperone protein or monosaccharide transferase (MST) and (b) a fluorescent protein, wherein at least one exon of a polynucleotide encoding a chaperone protein or MST is fused to a polynucleotide encoding a fluorescent protein.

2. The construct of claim 1, wherein the chaperone protein is ERp57.

3. The construct of claim 1, wherein the polynucleotide encodes green fluorescent protein.

4. The construct of claim 1, wherein (a) the chaperone protein is ERp57 and (ii) the polynucleotide encodes green fluorescent protein.

5. The construct of claim 1, wherein the fluorescent protein is selected from the group consisting of green fluorescent protein, red fluorescent protein, cyan fluorescent protein, yellow fluorescent protein, or mCherry.

6. A cell comprising the construct of claim 1.

7. The cell of claim 6, wherein the cell is a mammalian cell.

8. The cell of claim 7, wherein the mammalian cell is a human cell.

9. The cell of claim 7, wherein the mammalian cell is a stem cell.

10. The cell of claim 9, wherein the stem cell is an embryonic stem cell.

11. The cell of claim 6, wherein the mammalian cell is a neural cell.

12. The cell of claim 11, wherein the neural cell is a human neural cell.

13. (canceled)

14. A cellular model comprising a plurality of the cell according to claim 6.

15. A method of high throughput screening comprising a) administering at least one candidate therapeutic agent to the cell model of claim 14; and b) quantifying fluorescence.

16. The cellular model according to claim 14, wherein the cell is mammalian.

17. The cellular model according to claim 16, wherein the mammalian cell is human.

18. The cellular model according to claim 16, wherein the mammalian cell is a neural cell.

19. The cellular model according to claim 18, wherein the mammalian neural cell is human.

20. The method of claim 15, wherein fluorescence levels indicating a chaperone protein above 27 ng/ml identifies a potential therapeutic agent.
